(19) United States
(12) Patent Application Publication
(10) Pub. No.: US 2025/0169929 A1
(43) Pub. Date: May 29, 2025

(54) FIXING OF COLORING SOLUTIONS IN CERAMIC MULTI-COLOR INKJET 3D PRINTING

(71) Applicant: Ivoclar Vivadent AG, Schaan (LI)

(72) Inventors: Hendrik JOHN, Buchs (CH); Frank ROTHBRUST, Röns (AT); Sebastian KROLIKOWSKI, Richterswil (CH); Christian RITZBERGER, Grabs (CH)

(21) Appl. No.: 18/952,208

(22) Filed: Nov. 19, 2024

(30) Foreign Application Priority Data
Nov. 29, 2023 (EP) 23213076.5

(51) Int. Cl. A61C 13/00; A61C 13/08; B28B 1/00; B28B 11/00; B33Y 10/00; B33Y 30/00; B33Y 40/00; B33Y 80/00; C04B 41/00; C04B 41/45; C04B 41/50; C04B 41/52; C04B 41/89

(52) U.S. Cl. CPC A61C 13/0019; A61C 13/082; B28B 1/001; B28B 11/001; B33Y 10/00; B33Y 30/00; B33Y 40/00; B33Y 80/00; C04B 41/0072; C04B 41/009; C04B 41/4543; C04B 41/5012; C04B 41/52; C04B 41/89

(57) ABSTRACT

A method of producing a dental restoration by jet-printing, including jet-printing one or more layers of a dental restoration using a ceramic slurry, jet-printing a coloring solution onto the one or more layers, and applying a fixing solution for fixing the coloring solution.

### CROSS-REFERENCE TO RELATED APPLICATIONS

This application claims priority to European Patent Application No. 23213076.5 filed on Nov. 29, 2023, which is incorporated herein by reference in its entirety.

### TECHNICAL FIELD

The present invention relates to a method of producing a dental restoration by jet-printing and to a production apparatus for producing a dental restoration by jet-printing.

### BACKGROUND

When manually coloring ceramic restorations made of zirconium dioxide, infiltration solutions of nitrate salts are used for individual coloring. These are applied to the surface of the restoration using a brush, for example, and then diffuse into the porous ceramic. This application requires a high degree of experience on the part of the dental technician.

This coloring process of zirconium dioxide white bodies can be automated by using an airbrush system to infiltrate the restoration under computer control. In additive manufacturing processes, differently colored, solvent-based ceramic slurries can be selectively applied using an inkjet. Processing highly filled ceramic slurries places high demands on the print heads used in terms of particle size, filling level and therefore viscosity and abrasion. Jet-printing (jetting) of aqueous slurries is an alternative to solvent-based slurries. In this, the jetted material layer is dried without cracks.

In a manual infiltration process, however, the salt solutions diffuse into the zirconium dioxide white body in an uncontrolled manner. If the material is applied layer by layer in combination with selective coloring layer by layer of the previously applied ceramic layer, the previously deposited nitrate salts are dissolved again in the underlying layers and continue to diffuse uncontrolled into the already produced ceramic body.

US20200017699, 20120308837 and 20100249305 are directed to 3-D inkjet printing and are hereby incorporated by reference in their entirety.

### SUMMARY

It is the technical task of the present invention to prevent the uncontrolled diffusion or running of selectively applied nitrate salt solutions as coloring solutions during a spatial layer build-up process.

This task is solved by subject-matter according to the independent claims. Technically advantageous embodiments are the subject-matter of the dependent claims, the description and the drawings.

According to a first aspect, the technical task is solved by a method of producing a dental restoration by jet-printing, comprising the steps of jet-printing one or more layers of the dental restoration by means of a ceramic slurry; jet-printing a coloring solution onto the one or more layers; and applying a fixing solution for fixing the coloring solution. This can prevent the uncontrolled diffusion or running of selectively applied nitrate salt solutions as a coloring solution during a layer build-up process. Controlled, selective coloring of the 3D-printed green body using coloring solutions during the additive manufacturing process is ensured. Selective material application of the slurry and selective coloring are carried out separately. The same or different print heads can be used for material application and coloring. The fixing solution, for example, prevents diffusion of the coloring solution on the aqueous slurry to produce a multicolor green body. This ensures, for example, controlled, selective coloring of the 3D-printed ZrO₂ green body by means of coloring solutions during the additive manufacturing process.

In a technically advantageous embodiment of the method, the fixing solution is sprayed onto the one or more layers. This achieves the technical advantage, for example, that the fixing solution can be applied quickly and over a large area.

In a further technically advantageous embodiment of the method, the fixing solution is jet-printed onto the one or more layers by means of a print head. This achieves the technical advantage, for example, that the fixing solution can be selectively applied with pinpoint accuracy.

In a further technically advantageous embodiment of the method, the one or more layers are dried before or after jet-printing the coloring solution. This achieves the technical advantage, for example, that diffusion of the coloring solution is reduced.

In a further technically advantageous embodiment of the method, the one or more layers are dried before or after applying the fixing solution. This also achieves the technical advantage, for example, that diffusion of the coloring solution is reduced.

In a further technically advantageous embodiment of the method, a pH of the fixing solution is greater than 7. This achieves the technical advantage, for example, that running of the coloring solution is effectively reduced.

In a further technically advantageous embodiment of the method, the fixing solution is an ammonia-ammonium chloride solution. This achieves the technical advantage, for example, that the coloring solutions are fixed locally.

In a further technically advantageous embodiment of the method, the steps to produce the dental restoration are repeated. This achieves the technical advantage, for example, that the entire dental restoration can be built up and colored.

In a further technically advantageous embodiment of the method, the dental restoration is sintered in a sintering furnace. This achieves the technical advantage, for example, that a dental restoration with high strength can be produced.

According to a second aspect, the technical task is solved by a production apparatus for producing a dental restoration by jet-printing, comprising a first print head for jet-printing one or more layers of the dental restoration by means of a ceramic slurry; and a second print head for jet-printing a coloring solution onto the one or more layers; and an application device for applying a fixing solution for fixing the coloring solution. The production apparatus achieves the same technical advantages as the method according to the first aspect.

In a technically advantageous embodiment of the production apparatus, the application device is formed by a spray head for applying the fixing solution extensively. This achieves the technical advantage, for example, that the fixing solution can be applied quickly and over a large area.

In a further technically advantageous embodiment of the production apparatus, the application device is formed by a print head for selectively applying the fixing solution. This achieves the technical advantage, for example, that the fixing solution can be applied selectively with pinpoint accuracy.

In a further technically advantageous embodiment of the production apparatus, the production apparatus comprises a storage container for the fixing solution. The storage container can be replaceable. This achieves the technical advantage, for example, that the fixing solution can be stored and easily replaced.

In a further technically advantageous embodiment of the production apparatus, the production apparatus comprises a drying device for drying the applied materials, such as a slurry layer, coloring solution, fixing solution. This achieves the technical advantage, for example, that further diffusion can be reduced.

In a further technically advantageous embodiment of the production apparatus, the drying device comprises a blower and/or an infrared radiator. This achieves the technical advantage, for example, that the layers can be dried quickly.

According to a third aspect, the technical task is solved by a jet-printing system, comprising a production apparatus for producing a dental restoration by jet-printing; a coloring solution for printing onto one or more layers; and a fixing solution for fixing the coloring solution. The jet-printing system achieves the same technical advantages as the method according to the first aspect.

In a technically advantageous embodiment of the jet-printing system the coloring solution and/or the fixing solution is stored in a container. This achieves the technical advantage, for example, that the solutions can be stored reliably.

In a further technically advantageous embodiment of the jet-printing system the container is replaceable. This achieves the technical advantage, for example, that the solutions can be exchanged easily.

### BRIEF DESCRIPTION OF THE DRAWINGS

Exemplary embodiments of the invention are shown in the drawings and are described in more detail below, in which:

FIG. 1 shows an illustration of different regions of an incisor;

FIG. 2 shows a schematic illustration of a dental restoration;

FIG. 3 shows a schematic illustration of a production apparatus for producing a dental restoration;

FIG. 4 shows a schematic view of a method of producing a dental restoration by jet-printing;

FIG. 5 shows a schematic view of a further method of producing a dental restoration by jet-printing;

FIG. 6 shows a table with the compositions of different coloring solutions; and

FIG. 7 shows a block diagram of a method of producing a dental restoration.

### DETAILED DESCRIPTION

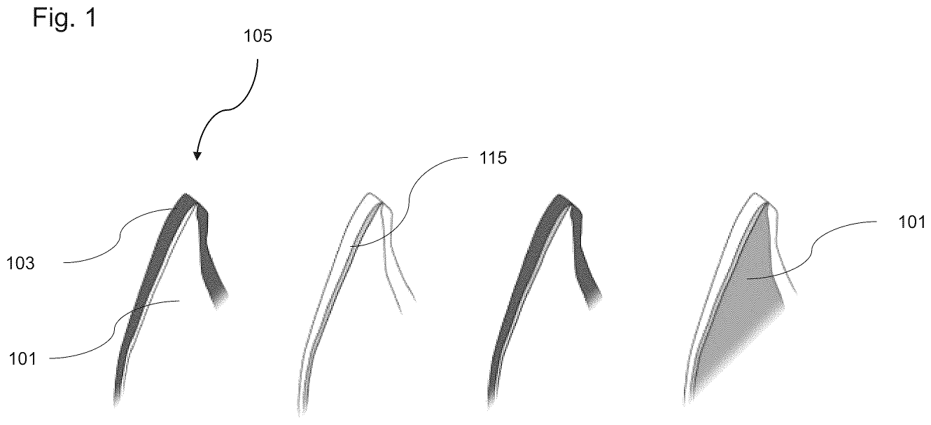

FIG. 1 shows an illustration of different regions of a tooth 105. The tooth 105 comprises an inner dentin core 101 and an outer enamel 103. The opaque, i.e., non-transparent, dentin core 101 is responsible for the basic coloration of the tooth 105. This shines through the enamel 103 of the incisal edge. The enamel 103 is naturally translucent. Translucency is the partial permeability to light of a body. In order to make a dental restoration look as lifelike as possible, this structure of the tooth 105 is also used in artificial dental restorations. For this purpose, materials with different optical properties are used in the production of the dental restoration.

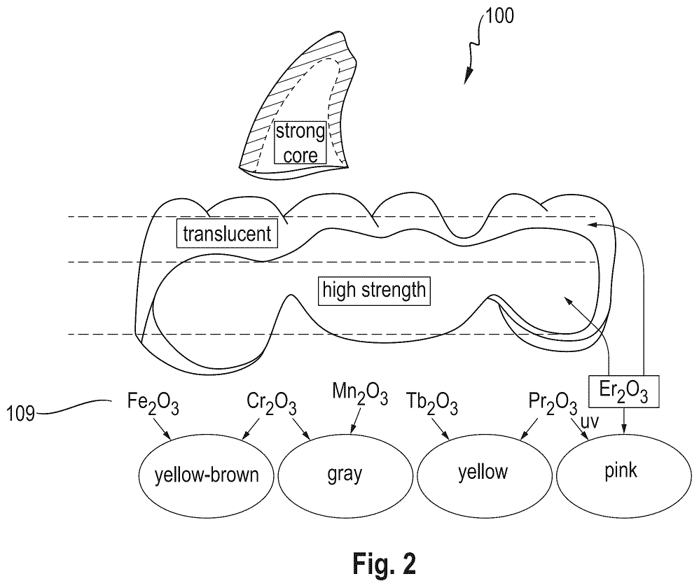

FIG. 2 shows a schematic illustration of a dental restoration 100. The dental restoration 100 serves as a dental prosthesis and is formed, for example, by a bridge, crown, full or partial prosthesis. The dental restoration 100 is selectively built up layer by layer, for example from 1-n different ceramic slurries 109 by means of a jet-printing process and, after selective coloring layer by layer using nitrate salts such as Fe₂O₃, Cr₂O₃, Mn₂O₃, Tb₂O₃, Pr₂O₃, Er₂O₃, CO₃O₄, NiO, TiO₂, CeO₂ and/or doping using e.g., yttrium and a final sintering of the white body of the dental restoration 100 produced in this way, a corresponding characterization with regard to color, translucency, opacity and mechanical properties is obtained.

In this process, the dental restoration 100 is built up by successive layers that are printed on top of each other. The ceramic powders of the slurries 109 can already be provided in predetermined translucencies. Mixing these slurries 109 results in the desired translucency of the dental restoration 100 in the respective spatial region.

After the selective material application of a layer of the slurry 109 by means of the jet-printing process, this layer is dried without cracks by evaporating the water or solvent as a binder. What remains is a porous white body layer with a layer thickness of 1 μm to 50 μm and a density of at least 2.5 g/cm³.

This process is repeated until the entire dental restoration 100 is built up spatially in layers.

In the case of Zro₂ slurries 109, different yttrium-doped ceramic powders (3 mol % yttrium-3Y-TZP, 4 mol % yttrium-4Y-TZP, 5 mol % yttrium-5Y-TZP) can also be used for the different strengths. In the process, the yttrium doping is also responsible for the degree of translucency. The different yttrium-doped ceramic powders have different properties.

$$3 Y ‐ TZP = low translucency / high strength \\ 4 Y ‐ TZP = medium translucency / medium strength \\ 5 Y ‐ TZP = high translucency / low strength$$

However, if different pre-colored slurries 109 are used, all of which also have different translucency and/or mechanical properties, each of these slurries 109 is assigned its own print head 111-1. If, for example, a general CYMK color scheme is used to cover the entire color space, 3×4=12 slurries 109 are contained in 12 print heads. The production of these different slurries 109 is also complex. This large number of slurries 109 is stored and maintained as different articles.

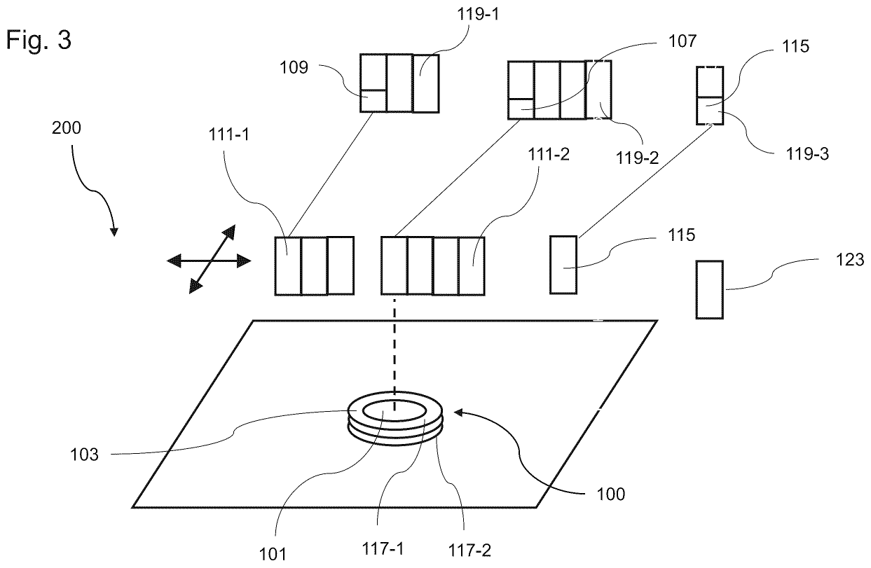

FIG. 3 shows a schematic illustration of a production apparatus 200 for producing a dental restoration 100 by jet-printing (inkjet-printing) of aqueous or solvent-based slurries 109. In order to additively manufacture ceramic, dental multi-material and multi-color restorations 100 by means of jet-printing, base slurries 109 are processed.

The production apparatus 200 comprises a plurality of storage containers 119-1 in which the base slurries 109 with different optical properties are arranged. Furthermore, the production apparatus 200 comprises at least several storage containers 119-2 in which the coloring solutions 107 are received. In addition, the production apparatus 200 comprises a storage container 119-3, in which a fixing solution 115 is received. The production apparatus for producing the dental restoration by jet-printing; the coloring solution for printing onto one or more layers; and the fixing solution for fixing the coloring solution form together a jet-printing system.

The ceramic slurries 109 are each applied drop by drop in several layers 117-1, . . . , 117-n by means of an assigned print head 111-1 in order to build up the dental restoration 100 spatially layer by layer. The print head 111-1 is movable in two directions so that the slurry 109 can be printed at any position. The print head(s) can also be arranged fixedly and the build platform moves in the three spatial directions below the print heads.

Slurries 109 with a droplet volume of typically 10 to 100 pL are used for selective material application, which eliminates the time-consuming debinding process. Electrically controlled piezo elements, for example, are used to eject the droplets.

A further print head 111-2 is used for applying the coloring solutions 107, through which the coloring solutions 107 can be jet-printed onto one or more layers 117-1, . . . , 117-n. The print head 111-2 is also movable in two directions, so that the coloring solutions 107 can be printed at any position. The print heads 111-1 and 111-2 can be controllable independently of each other or integrated in a common print module. The print heads 111-1 and 111-2 can also be arranged fixedly and the build platform moves in the three spatial directions below the print heads.

The fixing solution 115 is applied by a further application device 121, which may be formed by a spray head for applying the fixing solution 115 extensively or a further print head for selectively applying the fixing solution 115.

A drying device 123 is provided for drying the aqueous material, with which the layers 117-1, . . . , 117-n, the coloring solution 107 and the fixing solution 115 can be dried without cracks. The drying device 123 is formed, for example, by a blower and/or an infrared radiator.

The production apparatus 200 provides a reduced number of pre-colored and yttrium-doped neutral base slurries 109 to minimize the number of print heads 111-1 and 111-2 and still achieve an esthetic and functional result of the dental restoration 100. The coloring is done separately by the selective application of coloring solutions 107.

The desired tooth color is composed and mixed from the pre-colored coloring solutions 107. A subtractive color system is used, which spans a limited, dental color space (dental color gamut). The color mixing, the three-dimensional halftoning or dithering, of these pre-colored slurries 109 in various ratios is then created in the limited dental color space (gamut), which covers the common tooth colors, but not all colors.

Different coloring solutions 107 are selectively applied to a layer 117-1, . . . , 117-n using a 3D dithering algorithm, which is executed by a dithering module 113. For this purpose, the dithering module 113 comprises a processor for executing the 3D dithering algorithm and a digital data memory for storing the calculated mixing ratios and the dithering algorithm. The processor comprises any hardware system, component, or mechanism that processes data, signals, or other information. A processor may comprise a system with a central processing unit (CPU), several data processing units (MPU), dedicated electrical circuitry to achieve functionality, or other systems. The data storage may comprise hard disks, flash memory cards, random access memories (RAM) or read-only memories (ROM).

During dithering, the different coloring solutions are selectively applied in a certain ratio and two-dimensional printing pattern in the printing plane using the jet-printing process. The 3D dithering algorithm also calculates that two-dimensional dithering patterns that are not the same are applied in several layers 117-1, . . . , 117-n on top of each other. This prevents optical artifacts such as stripes or moiré patterns on vertical surfaces. The color mixing, three-dimensional halftoning or dithering of these pre-colored slurries 109 in various ratios is created within the limited dental color space (gamut), which covers the common tooth colors, but not all general colors.

When using highly viscous slurries 109 with a viscosity of more than 100 mPas with a high degree of filling, special requirements are placed on the print head 111-1 and its fluid system. The print head 111-1 is compatible with the binder used and/or the carrier material of the slurry 109 in order to avoid incompatibilities and consequences such as corrosion on the print head.

After the selective material application of a layer 117-1, . . . , 117-n of the slurry 109 by means of the jet-printing process, this layer 117-1, . . . , 117-n is dried without cracks by evaporating the water or the solvent as a binder. The ceramic powder of the slurry 109 can already be colored in a color, for example a base color or tooth color. What remains is a porous white body layer with a layer thickness of 1 μm to 50 μm and a density of at least 2.5 g/cm³. This process is repeated until the entire dental restoration 100 is built up spatially in layers.

If two slurries 109 with different translucencies are available, the high-strength and opaque slurry 109 can be used for the dentin core and the normal-strength and highly translucent slurry 109 for the incisal edge. The selective coloring is carried out after 1-n layers 117-1, . . . , 117-n of the material application by selectively jet-printing coloring solutions, such as nitrate solutions (acids), according to a corresponding color coding or color information.

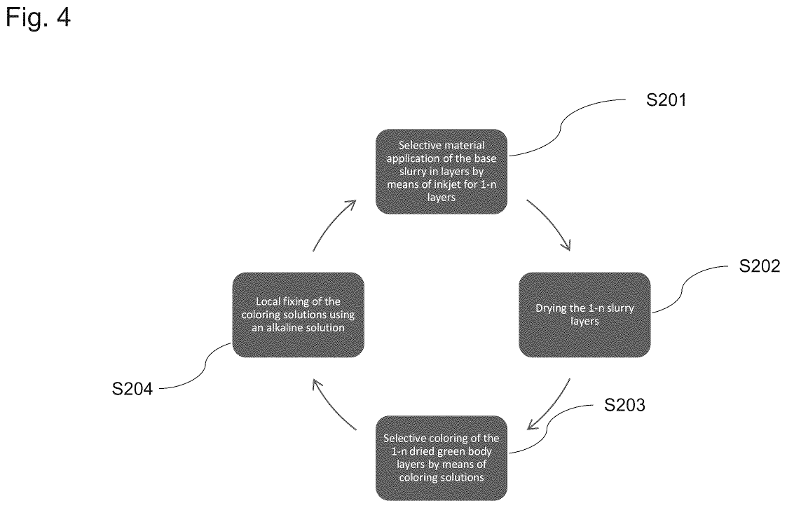

FIG. 4 shows a schematic view of a method of producing the dental restoration 100 by jet-printing. In step S201, a layer-by-layer selective material application of a slurry 109 is first carried out by means of a jet-printing process (inkjet process) for 1-n layers 117-1, . . . , 117-n.

In step S202, the applied slurry layer is dried, for example by supplying hot air. In step S203, the 1 to n applied layers 117-1, . . . , 117-n of the slurry 109 (green body layers) are selectively colored by means of coloring solutions. Here, the coloring solutions are also printed onto the 1 to n applied layers 117-1, . . . , 117-n by means of jet-printing. In step S204, the printed coloring solutions are locally fixed by means of a fixing solution, i.e. by means of an alkaline solution with a pH greater than 7 (lye). Steps S201 to S204 are then repeated until the dental restoration 100 is completely built up spatially.

For the method, either aqueous or solvent-based slurries 109 can be processed. Selective material application (step S201) and selective coloring (step S203) are carried out separately. The process has the technical advantage that with dried, i.e. anhydrous layers 117-1, . . . , 117-n, the pH of the coloring solution hardly plays a role.

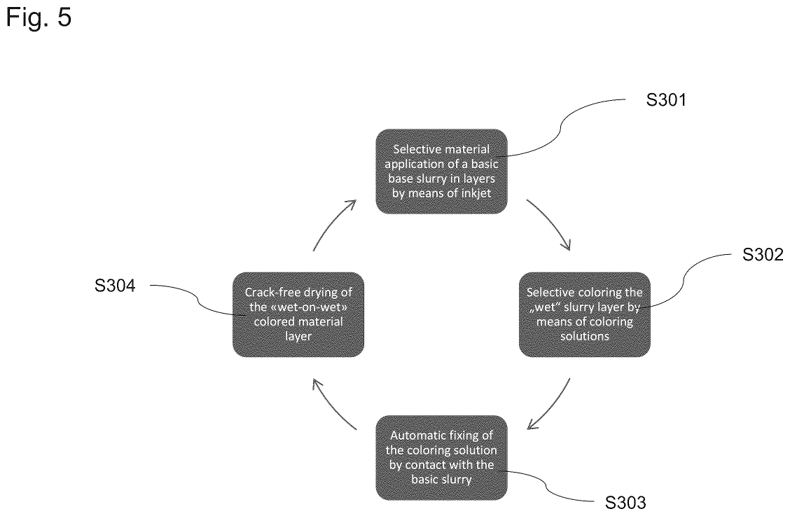

FIG. 5 shows a schematic view of a further method of producing the dental restoration 100 by jet-printing. In step S301, a layer-by-layer selective material application of a basic slurry 109 is first carried out by means of a jet-printing process (inkjet process) for 1-n layers.

The basic slurry 109 has a pH of greater than 7. A conventional basic slurry 109 contains H₂O, dispersing aids, such as 0 to 5% by weight, preferably 0.01 to 5% by weight of carboxylic acid derivatives (citric acid) or 0 to 5% by weight of ammonium polyacrylic acid (NH₄PAA), preferably 0.01 to 5% by weight. The pH (8-11) can be adjusted with NH₄OH. The basic adjustment of the slurry 109 has the advantage that the ceramic slurry 109 behaves less aggressively with regard to corrosion of the metal parts of the print head.

In step S302, the still wet and undried applied layers 117-1, . . . , 117-n of the slurry 109 (green body layers) are selectively colored by means of the coloring solutions. A precipitation reaction occurs as soon as the nitric acid (coloring solution) comes into contact with the wet slurry 109.

In step S303, the coloring solutions are automatically fixed by contact with the basic slurry 109. In step S304, the wet-on-wet colored layers 117-1, . . . , 117-n are dried without cracks, for example by supplying hot air. Steps S301 to S304 are then repeated until the dental restoration 100 is completely built up spatially.

For the method, either aqueous or solvent-based slurries 109 can be processed. Selective material application (step S301) and selective coloring (step S302) are carried out separately.

If no basic slurry 109 is used, the coloring solution 107 diffuses into the layers 117-1, . . . , 117-n. The applied coloring solution is fixed by separate application of the fixing solution, such as an alkaline solution.

During the process, the slurry 109 is stabilized in the acidic pH value range. After printing and drying the respective individual layers, a basic fixing solution is applied first, such as 0.1N ammonium hydroxide solution. The coloring solution 107 is then applied. When the coloring solution hits the layer treated with the fixing solution, the pH of the coloring solution is shifted locally towards pH>2. As the pH of the coloring solution is shifted, metal hydroxides, such as Fe(OH)₃, are formed locally at points where the coloring solution is to be fixed. This process is referred to as precipitation or flocculation.

After printing and drying the respective individual layers, the coloring solution can also be applied, followed immediately by the basic fixing solution, which in turn fixes the coloring components in the desired position.

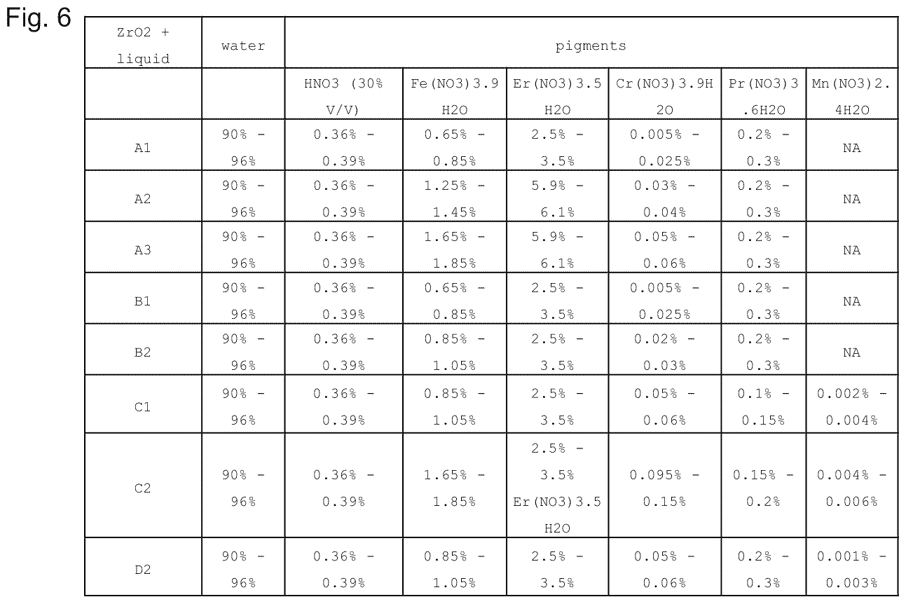

FIG. 6 shows a table with the compositions of different coloring solutions 107. The coloring solutions 107 are of low viscosity and can be jet-printed using either an inkjet-print head with DOD technology or bubble jet technology. Several color channels can be integrated in this print head for multi-color printing. The print head can apply the coloring solution to the layer 117-1, . . . , 117-n at a high resolution of 720 dpi.

With six available print heads, only one print head is used for medium translucency slurries 109 and one print head for support material. With seven available print heads, two print heads are respectively used for an opaque slurry 109 and a highly translucent slurry 109 and one print head is used for support material. The other four print heads are used for the coloring solutions 107.

The coloring solutions are nitrate salt or chloride salt solutions on an aqueous basis. Various metal salts (e.g. Fe(NO₃)_{3.9}H₂O; Pr(NO₃)_{3.6}H₂O; Tb(NO₃)_{3.5}H₂O; Er(NO₃)_{3.5}H₂O; Mn(NO₃)_{2.4}H₂O; Co(NO₃)_{2.6}H₂O; Cr(NO₃)_{3.9}H₂O; Mg(NO₃)_{2.6}H₂O; Al(NO₃)_{3.9}H₂O; Cu(NO₃)_{2.3}H₂O; Zn(NO₃)_{2.6}H₂O; Y(NO₃)_{3.6}H₂O; La(NO₃)_{3.6}H₂O; Ce(NO₃)_{3.6}H₂O; Nd(NO₃)_{3.6}H₂O; Sm(NO₃)_{3.6}H₂O; Gd(NO₃)_{3.6}H₂O; Yb(NO₃)_{3.6}H₂O; Ni(NO₃)_{2.6}H₂O; Co(NO₃)_{2.6}H₂O; Ga(NO₃)₃·XH₂O; In(NO₃)₃·XH₂O) are dissolved in different concentrations.

However, the coloring solutions 107 can also be significantly more concentrated depending on the desired composition. However, increasing the ion concentration increases a viscosity or shifts the pH value to the acidic range (<7).

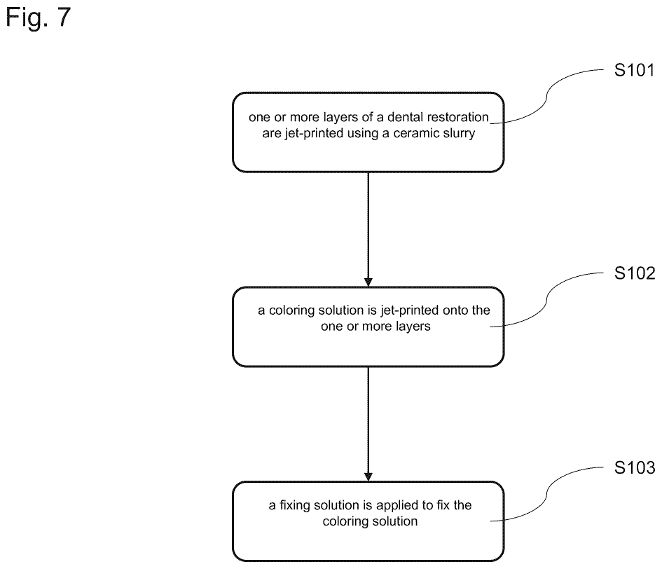

FIG. 7 shows a block diagram of a method of producing a dental restoration 100 by jet-printing. In step S101, the layers 117-1, . . . , 117-n of the dental restoration 100 are jet-printed by means of the ceramic slurry 109. In step S102, the coloring solution 107 is jet-printed onto the one or more layers 117-1, . . . , 117-n. Then, in step S103, the fixing solution 115 is applied to fix the coloring solution 107. These steps are repeated until the dental restoration 100 is completely built up.

If the coloring solution 107 is jet-printed into the previously dried layer 117-1, . . . , 117-n and then moist slurry is applied again to the previous, dried and colored layer 117-1, . . . , 117-n, it may happen that the coloring solution 107 is dissolved again and diffuses uncontrollably in the already built-up dental restoration 100. When jet-printing the coloring solution 107 onto the dried layer 117-1, . . . , 117-n, care must be taken to ensure that the penetration depth of the coloring solution 107 can be controlled. If, for example, the infiltration depth is greater than a layer thickness, the coloring can only take place after multiple layers of slurry 109 have been applied.

By applying the fixing solution 115, the coloring solution 107 is locally fixed so that uncontrolled diffusion or mixing is prevented by precipitation of the hydroxides. This can be done by applying a basic fixing solution 115 over a large area or selectively, such as, but not limited to, an ammonia-ammonium chloride buffer solution, to shift the pH. For example, the ammonia-ammonium chloride buffer solution comprises 987.8 g of water, 6.8 g of 25%-ammonia and 5.4 g of ammonium chloride.

A single slurry 109 with a basic coloration and a medium translucency can be used for the additive material application of the dental restoration 100. The selective coloring of the slurry 109 is achieved by jet-printing the coloring solutions, which are then fixed.

Different print head technologies can be used, as the slurries 109 and the coloring solution 107 have different rheological properties. The slurry 109 is highly viscous (10-1000 mPas depending on the shear rate) and contains a high weight/volume proportion of an abrasive filler in the form of ceramic particles. A low viscosity of the slurry 109 between 10-500 mPas is advantageous and a viscosity between 10-100 mPas is even more advantageous. The higher the viscosity of the slurry 109, the more difficult it is to process using inkjet-print heads. The print head should also be able to jet aqueous media.

The coloring solution 107, on the other hand, has a low viscosity (0.5-50 mPas). As these are highly concentrated nitrate solutions, the print head used should be resistant to nitrates. In addition, the volumes processed in each case are different and are in a volume ratio of approx. 98% for the slurry 109 and 2% for the coloring solution 107. The selective application of coloring solutions 107 can also be carried out using a multi-channel print head 111-2.

The penetration or diffusion depth of the coloring solution 107 can also be controlled by adjusting the viscosity. This is done by adding a suitable thickening agent to the coloring solution 107, which is stable in the pH range of the coloring solution, such as polyvinyl pyrolidone (PVP).

The main volume is processed via the slurry 109, whereas less volume is processed by the coloring solution 107. The coloring solution 107 and the slurry 109 can be stored in differently sized storage containers 119-1 and 119-2.

The fully built-up dental restoration 100 is then sintered in a sintering furnace. The slurry 109 and the coloring solutions 107 can be adjusted to a uniform sintering behavior. The sintering kinetics can be homogeneously adjusted by adjusting the individual coloring solutions 107. The sintering activators are “neutralized” by the use or addition of sintering inhibitors. Examples of sintering inhibitors and activators are set forth in U.S. Pat. No. 11,413,122, which is hereby incorporated by reference in its entirety.

Nitrate solutions can be used as coloring solution 107 for the selective coloring process, regardless of whether the slurries 109 are already pre-colored or the coloring is carried out exclusively by the layer-by-layer selective application of the coloring solutions 107 during the additive, layer-by-layer production process.

In addition, the opacity of the slurry 109 of the dental restoration 100 can be adjusted using variable components of yttrium or ytterbium in the coloring liquid. For example, the elements yttrium, ytterbium, neodymium and europium are used as translucency enhancers and aluminum and silicon as opaque liquids. The yttrium content can be increased with a suitable coloring solution. In this way, different opacity values can be achieved in the dental restoration 100, such as opaque for the dentin core and translucent for the incisal edge.

All the features explained and shown in connection with individual embodiments of the invention can be provided in different combinations in the subject matter according to the invention in order to simultaneously realize their advantageous effects.

All method steps can be implemented by devices that are suitable for executing the respective method step. All functions performed by the features of the subject matter can be a method step of a method.

The scope of protection of the present invention is given by the claims and is not limited by the features explained in the description or shown in the figures.

### REFERENCE LIST

100 Dental restoration
101 Dentin core
103 Enamel
105 Tooth
107 Coloring solution
109 Slurry
111 Print head
113 Dithering module
115 Fixing solution
117 Layer
119 Storage container
121 Application device
123 Drying device

1. A method of producing a dental restoration by jet-printing, comprising
jet-printing one or more layers of the dental restoration using a ceramic slurry;
jet-printing a coloring solution onto the one or more layers; and
applying a fixing solution for fixing the coloring solution.

2. The method according to claim 1,
wherein the fixing solution is sprayed onto the one or more layers.

3. The method according to claim 1,
wherein the fixing solution is jet-printed onto the one or more layers by a print head.

4. The method according to claim 1,
wherein the one or more layers are dried before or after jet-printing the coloring solution.

5. The method according to claim 1,
wherein the one or more layers are dried before or after applying the fixing solution.

6. The method according to claim 1,
wherein a pH of the fixing solution is greater than 7.

7. The method according to claim 1,
wherein the fixing solution is an ammonia-ammonium chloride solution.

8. The method according to according to claim 1,
wherein the steps to produce the dental restoration are repeated.

9. The method according to claim 1,
wherein the dental restoration is sintered in a sintering furnace.

10. A production apparatus for producing a dental restoration by jet-printing, comprising:
a first print head for jet-printing one or more layers of the dental restoration by means of a ceramic slurry; and
a second print head for jet-printing a coloring solution onto the one or more layers; and
an application device for applying a fixing solution for fixing the coloring solution.

11. The production apparatus according to claim 10,
wherein the application device is formed by a spray head for applying the fixing solution extensively.

12. The production apparatus according to claim 10,
wherein the application device is formed by a print head for selectively applying the fixing solution.

13. The production apparatus according to claim 10,
wherein the production apparatus comprises a storage container for the fixing solution.

14. The production apparatus according to claim 10,
wherein the production apparatus comprises a drying device for drying the applied materials.

15. The production apparatus according to claim 10,
wherein the drying device comprises a blower and/or an infrared radiator.

16. A jet-printing system, comprising:
a production apparatus for producing a dental restoration by jet-printing;
a coloring solution for printing onto one or more layers; and
a fixing solution for fixing the coloring solution.

17. The jet-printing system according to claim 16,
wherein the coloring solution and/or the fixing solution is stored in a container.

18. The jet-printing system according to claim 17,
wherein the container is replaceable.
